(19) United States
(12) Patent Application Publication
(10) Pub. No.: US 2002/0002286 A1
(43) Pub. Date: Jan. 3, 2002

(54) Solid-phase process for making indole compounds

(76) Inventors: Daniel Michael Ketcha, Cincinnati, OH (US); Lawrence Joseph Wilson, Raritan, NJ (US)

Correspondence Address: THE PROCTER & GAMBLE COMPANY, PATENT DIVISION, HEALTH CARE RESEARCH CENTER, 8340 MASON-MONTGOMERY ROAD, MASON, OH, 45040, US

(21) Appl. No.: 09/779,080

(22) Filed: Feb. 8, 2001

Related U.S. Application Data
(60) Provisional application No. 60/181,155, filed on Feb. 9, 2000.

(51) Int. Cl. C 07D 2 9/14; C07F 9/547

(52) U.S. Cl. 548/413; 548/508; 548/494

(57) ABSTRACT

The subject invention involves a solid-phase process for the synthesis of indole compounds comprising the steps:

(a) treating an amino resin with diketene to provide a resin-bound acetoacetamide;

(b) treating the product from Step (a) with a primary amine in the presence of a dehydrating agent to provide a resin-bound enaminone;

(c) treating the product from Step (b) with a 1,4-benzoquinone, and releasing the resulting indole product from the resin.

### CROSS REFERENCE

This application claims priority under Title 35, United States Code 119(e) from Provisional Application Ser. No. 60/181,155, filed Feb. 9, 2000.

### FIELD OF THE INVENTION

The subject invention involves a solid-phase process for the synthesis of indole compounds using a Nenitscu reaction.

### BACKGROUND OF THE INVENTION

The remarkable range of biological activities exhibited by indole containing molecules provides compelling impetus for development of new and improved methods for the construction of this heterocycle and the need to adapt known ring-forming reactions to the solid-phase. Despite the extensive number of preparative methods available for the synthesis of the indole ring, to date solid-phase methodologies have been limited.

### SUMMARY OF THE INVENTION

The subject invention involves a solid-phase process for the synthesis of indole compounds comprising the steps:

(a) treating an amino resin with diketene to provide a resin-bound acetoacetamide:

[chemical structure]

(b) treating the resin-bound acetoacetamide from Step (a) with a primary amine, R-NH2, in the presence of a dehydrating agent to provide a resin-bound enaminone:

[chemical structure]

wherein R is selected from the group consisting of alkyl, aryl, and heterocyclyl;

(c) treating the resin-bound enaminone from Step (b) with a 1,4-benzoquinone:

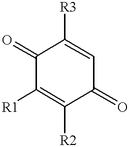

to provide resin-bound product:

[chemical structure]

wherein each of R1,R2 R3 are independently selected from the group consisting of hydrogen, halo, nitro, cyano, alkyl, aryl, heterocyclyl, and any of the following unsubstituted or substituted with alkyl, aryl or heterocyclyl: hydroxy, thio, amino, formyl, formoyl, formylamino, aminoformyl, sulfo, sulfonyl, phospho, phosphonyl, carboxy;

(d) optionally treating the resin-bound product from Step (c) with an alcohol, R4-OH, or a halide, R4-Cl, to provide resin-bound product:

[chemical structure]

wherein R4 is alkyl;

(e) treating the resin-bound product from Step (c) or from Step (d) to release indole product from the resin:

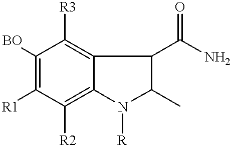

wherein B is H or R4, respectively.

### DETAILED DESCRIPTION OF THE INVENTION

As used herein unless specified otherwise, “alkyl” means a hydrocarbon chain which is branched, linear or cyclic, saturated or unsaturated (but not aromatic), substituted or unsubstituted. The term “alkyl” may be used alone or as part of another word where it may be shortened to “alk” (e.g., in alkoxy, alkylacyl). Preferred linear alkyl have from one to about twenty carbon atoms, more preferably from one to about ten carbon atoms, more preferably still from one to about six carbon atoms, still more preferably from one to about four carbon atoms; most preferred are methyl or ethyl. Preferred cyclic and branched alkyl have from three to about twenty carbon atoms, more preferably from three to about ten carbon atoms, more preferably still from three to about seven carbon atoms, still more preferably from three to about five carbon atoms. Preferred cyclic alkyl have one hydrocarbon ring, but may have two, three, or more, fused or spirocycle hydrocarbon rings. Preferred alkyl are unsaturated with from one to about three double or triple bonds, preferably double bonds; more preferably they are mono-unsaturated with one double bond. Still more preferred alkyl are saturated. Saturated alkyl are referred to herein as “alkanyl”. Alkyl unsaturated only with one or more double bonds (no triple bonds) are referred to herein as “alkenyl”. Preferred substituents of alkyl include halo, alkyl, aryl, heterocycle, hydroxy, alkoxy, aryloxy, thio, alkylthio, arylthio, amino, alkylamino, arylamino, amide, alkylamide, arylamide, formyl, alkylacyl, arylacyl, carboxy and its alkyl and aryl esters and amides, nitro, and cyano. Also, unsubstituted alkyl are preferred.

As used herein, “heteroatom” means a nitrogen, oxygen, or sulfur atom.

As used herein, “alkylene” means an alkyl which connects two other moieties, “heteroalkylene” means an alkylene having one or more heteroatoms in the connecting chain.

As used herein unless specified otherwise, “aryl” means an aromatic hydrocarbon ring (or fused rings) which is substituted or unsubstituted. The term “aryl”may be used alone or as part of another word (e.g., in aryloxy, arylacyl). Preferred aryl have from six to about fourteen, preferably to about ten, carbon atoms in the aromatic ring(s), and a total of from about six to about twenty, preferably to about twelve, carbon atoms. Preferred aryl is phenyl or naphthyl; most preferred is phenyl. Preferred substituents of aryl include halo, alkyl, aryl, heterocycle, hydroxy, alkoxy, aryloxy, thio, alkylthio, arylthio, amino, alkylamino, arylamino, amide, alkylamide, arylamide, formyl, alkylacyl, arylacyl, carboxy and its alkyl and aryl esters and amides, nitro, and cyano. Also, unsubstituted aryl are preferred.

As used herein unless specified otherwise, “heterocycle” or “heterocyclyl” means a saturated, unsaturated or aromatic cyclic hydrocarbon ring (or fused rings) with one or more heteroatoms in the hydrocarbon ring(s). Preferred heterocyclyls are attached to the other structures at a carbon of the heterocyclyl ring. Preferred heterocycles have from one to about six heteroatoms in the ring(s), more preferably one or two or three heteroatoms in the ring(s). Preferred heterocyclyls have from three to about fourteen, preferably to about ten, carbon plus heteroatoms in the ring(s), more preferably from three to about seven, more preferably still five or six, carbon plus heteroatoms in the rings(s); and a total of from three to about twenty carbon plus heteroatoms, more preferably from three to about ten, more preferably still five or six, carbon plus heteroatoms. Preferred heterocyclyls have one ring, but may have two, three, or more, fused or spirocycle rings. More preferred heterocyclyl rings include those which are one ring with 5 or 6 carbon plus heteroatoms in the ring with one to about three ring heteroatoms, no more than two of which are O and S; and those which are two fused rings with 8-10 carbon plus heteroatoms in the rings with one to about four ring heteroatoms, no more than two of which are O and S. Still more preferred are such 5- or 6-ring atom heterocyclyls with one or two ring atoms being O or S and the others being C; or with one, two or three ring atoms being N and the others being C. Such preferred 5- or 6-ring atom heterocyclyls are preferably saturated, unsaturated with one or two double bonds, or aromatic. Such preferred 5- or 6-ring atom heterocyclyls are preferably a single ring; or fused with a 3- to 6-ring atom hydrocarbon ring which is saturated, unsaturated with one double bond, or aromatic (phenyl); or fused with another such 5- or 6-ring atom heterocyclic ring. Heterocyclyls are unsubstituted or substituted. Preferred heterocyclyl substituents are the same as for alkyl.

As used herein unless specified otherwise, “heteroaryl” means an aromatic heterocyclyl.

The subject invention process uses an amino resin. Such resins are well known and commercially available. The amino moieties of such commercially-available resins are generally protected by one of a number of well known protecting groups such as Fmoc, Boc, Cbz, TEOC, Alloc, Ddz. Such resins are designated herein by the structure:

[chemical structure]

wherein Pro is a protecting group.

Preferred resins used for the subject invention process include those having the following structure:

[chemical structure]

wherein each A is independently H or OMe, Y is H or 2,4-(MeO)₂Ph, Z is nil or C(O)O or C(O)NH, Q is nil or alkyl or aryl, and Pro is a protecting group. Particularly preferred resins are ArgoPORE macro-porous or polyethylene glycol based resins from Argonant Technologies, Inc. Especially preferred is highly crosslinked ArgoPore-Rink-NH-Fmoc resin:

[chemical structure]

If the amino moieties of the resin are protected, the initial step of the subject process is to remove the protecting group using well known conditions. Fmoc is typically removed by using peperidine in dimethlylformamide (DMF).

The next step is to acylate the amino groups of the resin by treatment with diketene 1 to produce resin-bound acetoacetamide 2:

[chemical structure]

This step is preferably carried out in dichloromethane (DCM) solvent at a temperature of from about −15° C. to about room temperature for a period of from about 1 to about 24 hours.

The next step of the subject process is to treat resin product 2 with a primary amine in the presence of trimethylorthoformate (TMOF) as a dehydrating agent to produce resin-bound enaminone 3:

[chemical structure]

wherein R is selected from alkyl, aryl, and heterocyclyl. Preferably R is selected from unsubstituted and substituted C₁ to about C₈ alkyl, unsubstituted or substituted phenyl or naphthyl, unsubstituted or substituted heterocyclyl having one ring or two fused rings with 5 or 6 ring atoms each. This step is preferably carried out in TMOF solvent at about room temperature for a period of from about 2 h to about 24 h, the TMOF also acting as the dehydrating agent.

The next step of the subject process is to treat resin product 3 with a 1,4-benzoquinone 4 to produce resin-bound product 5:

[chemical structure]

wherein each of R1, R2, and R3 is independently selected from hydrogen, halo, nitro, cyano, alkyl, aryl, heterocyclyl, and any of the following unsubstituted or substituted with alkyl, aryl or heterocyclyl: hydroxy, thio, amino, formyl, formoyl, formylamino, aminoformyl, sulfo, sulfonyl, phospho, phosphonyl, carboxy. Preferred R1, R2 and R3 are each independently selected from 10 hydrogen, unsubstituted or substituted C₁ to about C₈ alkyl, unsubstituted or substituted phenyl or naphthyl, unsubstituted or substituted heterocyclyl having one ring or two fused rings with 5 or 6 carbon plus heteroatoms in each ring, halo, C₁ to about C₈ alkoxy, C₁ to about C₈ alkyl esters of carboxy. This step is preferably carried out in a highly polar solvent, preferably in a solvent selected from nitromethane, acetone, acetic acid, dimethylsulfoxide, dimethylformamide. This step is preferably carried out at about room temperature for a period of about 24 to about 48 hours in nitromethane solvent.

The —OH on the phenyl ring of resin-bound product 5 can optionally be alkylated by treating with an alcohol (R4-OH) in the presence of triphenylphosphine (PPh₃) and diethylazodicarboxylate (DEAD) or with an alkyl halide (R4-X) in base:

[chemical structure]

wherein R4 is alkyl. Preferred R4 is C₁ to about C₁₆ alkyl, unsubstituted or substituted with alkyl, aryl, heterocyclyl. When the reactant is an alcohol, this step is preferably carried out in tetrahydrofuran (THF) solvent at a temperature of from about 0° C. to about 25° C. for a period of from about 1 to about 24 hours. When the reactant is alkyl halide, this step is preferably carried out in dimethylsulfoxide (DMSO) solvent at a temperature of from about 0° C. to about 25° C. for a period of from about 1 to about 24 hours.

The next step of the subject process is to treat resin-bound product 5 or 6 to release the corresponding indole product 7 from the resin by any known method:

[chemical structure]

wherein B is H or R4, respectively. This step is preferably carried out by treating 5 or 6 with about 20% trifluoroacetic acid (TFA) in DCM solvent at a temperature of from about 0° C. to about 25° C. for a period of from about 1 to about 4 hours.

The following are non-limiting examples of the preparation of indole compounds using the subject invention process.

[chemical structure]

As depicted in Scheme 1, deprotection (piperidine/DMF) of ArgoPore-Rink-NH-Fmoc resin 1 followed by treatment with diketene (DCM, −15° C. to rt) results in complete acylation of the amino groups (ninhydrin test) to provide the resin-bound acetoacetamide 2. Treatment of resin 2 with a variety of primary amines in the presence of TMOF as dehydrating agent then leads to formation of the resin-bound enaminones 3. Addition of 1,4-benzoquinones 4 in an appropriate solvent at room temperature (24-48 h) results in the formation of the characteristic brick-red color typically observed in this reaction. Trifluoroacetic acid (20%TFA/DCM) induces cleavage from the resin affording the appropriately substituted 5-hydroxyindole-3-carboxamides 8a-o (Table 1).

TABLE 1

| Exam- | | | |
|---|---|---|---|
| ple | R | Quinone (4) | R1, R2, R3 |
| 8a | PhCH₂— | 1,4-benzoquinone | H, H, H |
| 8b | PhCH₂— | 2-methyl-1,4-benzoquinone | CH₃, H, H |
| 8c | PhCH₂— | 2-methoxy-1,4- | CH₃O, H, H |
| | | benzoquinone | |
| 8d | PhCH₂— | 2-phenyl-1,4-benzoquinone | Ph, H, H |
| 8e | PhCH₂— | 2-chloro-1,4-benzoquinone | Cl, H, H |
| 8f | PhCH₂— | 2-bromo-1,4-benzoquinone | Br, H, H |
| 8g | PhCH₂— | 2-chloro-5-methyl-1,4- | H, CH₃, Cl |
| | | benzoquinone | |
| 8h | PhCH₂— | 1,4-naphthoquinone hydrate | (CH)₄, H |
| 8i | p-CH₃C₆H₄CH₂— | 1,4-benzoquinone | H, H, H |
| 8j | p-CH₃OC₆H₄CH₂— | 1,4-benzoquinone | H, H, H |
| 8k | p-CF₃C₆H₄CH₂— | 1,4-benzoquinone | H, H, H |
| 8l | p-ClC₆H₄CH₂— | 1,4-benzoquinone | H, H, H |
| 8m | m-ClC₆H₄CH₂— | 1,4-benzoquinone | H,H,H |
| 8n | (-)-PhCH(CH₃)— | 1,4-benzoquinone | H, H, H |
| 8o | CH₃(CH₂)₅— | 1,4-benzoquinone | H, H, H |

In Examples 8a-o, nitromethane is superior to acetone as a reaction solvent except for Examples 8d and 8e, where products of higher purity but lower yield are obtained using acetone. While in principle, three isomeric 5-hydroxyindole-3-carboxamides (e.g., substituents at 4-, 6-and 7-positions) are possible from the reaction of monosubstituted quinones with enaminones 3, regioselective formation of the C-6 isomer typically occurs. Utilization of 2-chloro-5-methylbenzoquinone typically results in clean conversion to the 4-chloro-7-methyl isomer as a result of enamine condensation ortho to the chlorine substituent. Naphthoquinone (Example h) affords a good yield of the necessarily 6,7-fused ring adduct. This solid-phase reaction is tolerant of a variety of substituents on the phenyl moiety of the benzylic amine component (Examples 8i-m), as well as branching as in the case of α-methylbenzylamine (Example 8n). Aliphatic amine n-hexylamine (Example 8o) provides the corresponding N-alkylindole.

The subject invention also includes libraries of compounds made by the subject invention process. Such libraries are mixtures of compounds or collections of isolated compounds of structure 7, wherein R, R1, R2, R3, R4 and B are as defined herein.

While particular embodiments of the subject invention have been described, it will be obvious to those skilled in the arts that various changes and modifications of the subject invention can be made without departing from the spirit and scope of the invention. It is intended to cover, in the appended claims, all such modifications that are within the scope of this invention.

What is claimed is:

1. A solid-phase process for the synthesis of indole compounds comprising the steps:
(a) treating an amino resin with diketene to provide a resin-bound acetoacetamide: [chemical structure]
(b) treating the resin-bound acetoacetamide from Step (a) with a primary amine, R-NH2, in the presence of a dehydrating agent to provide a resin-bound enaminone: [chemical structure]
wherein R is selected from the group consisting of alkyl, aryl, and heterocyclyl;
(c) treating the resin-bound enaminone from Step (b) with a 1,4-benzoquinone: 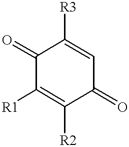
to provide resin-bound product: [chemical structure]
wherein each of R1, R2 and R3 are independently selected from the group consisting of hydrogen, halo, nitro, cyano, alkyl, aryl, heterocyclyl, and any of the following unsubstituted or substituted with alkyl, aryl or heterocyclyl: hydroxy, thio, amino, formyl, formoyl, formylamino, aminoformyl, sulfo, sulfonyl, phospho, phosphonyl, carboxy;
(d) optionally treating the resin-bound product from Step (c) with an alcohol, R4-OH, or a halide, R4-Cl, to provide resin-bound product: [chemical structure]
wherein R4 is alkyl;
(e) treating the resin-bound product from Step (c) or from Step (d) to release indole product from the resin: 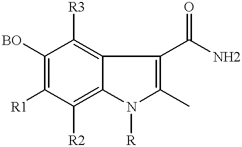
wherein B is H or R4, respectively.

2. The process of claim 1 wherein Step (c) is carried out in a highly polar solvent.

3. The process of claim 2 wherein the solvent in which Step (c) is carried out is selected from the group consisting of nitromethane, acetone, acetic acid, dimethylsulfoxide, and dimethylformamide.

4. The process of claim 3 wherein Step (c) is carried out at about room temperature for from about 24 to about 48 hours in nitromethane solvent.

5. The process of claim 1 wherein the dehydrating agent in Step (b) is TMOF.

6. The process of claim 3 wherein the dehydrating agent in Step (b) is TMOF.

7. The process of claim 4 wherein Step (b) is carried out at about room temperature for from about 2 to about 24 hours in TMOF solvent.

8. The process of claim 7 wherein Step (a) is carried out at a temperature of from about −15° C. to about room temperature for from about 1 to about 24 hours in DCM solvent.

9. The process of claim 1 wherein in Step (e), the resin-bound product is treated with TFA in DCM solvent.

10. The process of claim 5 wherein in Step (e), the resin-bound product is treated with TFA in DCM solvent.

11. The process of claim 7 wherein in Step (e), the resin-bound product is treated with about 20% TFA in DCM solvent at a temperature of from about 0° C. to about 25° C. for from about 1 to about 4 hours.

12. The process of claim 8 wherein in Step (e), the resin-bound product is treated with about 20% TFA in DCM solvent at a temperature of from about 0° C. to about 25° C. for from about 1 to about 4 hours.

13. The process of claim 1 wherein optional Step (d) is not carried out.

14. The process of claim 5 wherein optional Step (d) is not carried out.

15. The process of claim 12 wherein optional Step (d) is not carried out.

16. The process of claim 12 wherein Step (d) is carried out at a temperature of from about 0° C. to about 25° C. for from about 1 to about 24 hours in THF or DMSO solvent.

17. The process according to claim 1 wherein:
(a) R is selected from the group consisting of unsubstituted and substituted C₁ to about C₈ alkyl, unsubstituted or substituted phenyl or naphthyl, unsubstituted or substituted heterocyclyl having one ring or two fused rings with 5 or 6 ring atoms each;
(b) R1, R2 and R3 are each independently selected from the group consisting of unsubstituted or substituted C₁ to about C₈ alkyl, unsubstituted or substituted phenyl or naphthyl, unsubstituted or substituted heterocyclyl having one ring or two fused rings with 5 or 6 ring atoms each, halo, C₁ to about C₈ alkoxy, C₁ to about C₈ alkyl esters of carboxy;
(c) R4 is C₁ to about C₁₆ alkyl unsubstituted or substituted with alkyl, aryl or heterocyclyl.

18. The process according to claim 5 wherein:
(a) R is selected from the group consisting of unsubstituted and substituted C₁ to about C₈ alkyl, unsubstituted or substituted phenyl or naphthyl, unsubstituted or substituted heterocyclyl having one ring or two fused rings with 5 or 6 ring atoms each;
(b) R1, R2 and R3 are each independently selected from the group consisting of unsubstituted or substituted C₁ to about C₈ alkyl, unsubstituted or substituted phenyl or naphthyl, unsubstituted or substituted heterocyclyl having one ring or two fused rings with 5 or 6 ring atoms each, halo, C₁ to about C₈ alkoxy, C₁ to about C₈ alkyl esters of carboxy;
(c) R4 is C₁ to about C₁₆ alkyl unsubstituted or substituted with alkyl, aryl or heterocyclyl.

19. The process according to claim 15 wherein:
(a) R is selected from the group consisting of unsubstituted and substituted C₁ to about C₈ alkyl, unsubstituted or substituted phenyl or naphthyl, unsubstituted or substituted heterocyclyl having one ring or two fused rings with 5 or 6 ring atoms each;
(b) R1, R2 and R3 are each independently selected from the group consisting of unsubstituted or substituted C₁ to about C₈ alkyl, unsubstituted or substituted phenyl or naphthyl, unsubstituted or substituted heterocyclyl having one ring or two fused rings with 5 or 6 ring atoms each, halo, C₁ to about C₈ alkoxy, C₁ to about C₈ alkyl esters of carboxy;
(c) R4 is C₁ to about C₁₆ alkyl unsubstituted or substituted with alkyl, aryl or heterocyclyl.

20. The process according to claim 16 wherein:
(a) R is selected from the group consisting of unsubstituted and substituted C₁ to about C₈ alkyl, unsubstituted or substituted phenyl or naphthyl, unsubstituted or substituted heterocyclyl having one ring or two fused rings with 5 or 6 ring atoms each;
(b) R1, R2 and R3 are each independently selected from the group consisting of unsubstituted or substituted C₁ to about C₈ alkyl, unsubstituted or substituted phenyl or naphthyl, unsubstituted or substituted heterocyclyl having one ring or two fused rings with 5 or 6 ring atoms each, halo, C₁ to about C₈ alkoxy, C₁ to about C₈ alkyl esters of carboxy;
(c) R4 is C₁ to about C₁₆ alkyl unsubstituted or substituted with alkyl, aryl or heterocyclyl.
